(19) United States
(12) Patent Application Publication
(10) Pub. No.: US 2025/0160764 A1
(43) Pub. Date: May 22, 2025

(54) X-RAY IMAGING APPARATUS

(71) Applicants: RAYENCE Co., Ltd., Gyeonggi-do (KR); VATECH EWOO Holdings Co., Ltd., Gyeonggi-do (KR); Qpix solutions Inc., Morrisville, NC (US)

(72) Inventors: Seungman YUN, Morrisville, NC (US); Changhyeuk KIM, Morrisville, NC (US); Byeongnyeon KIM, Gyeonggi-do (KR); Kyuseok PARK, Gyeonggi-do (KR)

(73) Assignees: RAYENCE Co., Ltd., Gyeonggi-do (KR); VATECH EWOO Holdings Co., Ltd., Gyeonggi-do (KR); Qpix solutions Inc., Morrisville, NC (US)

(21) Appl. No.: 18/952,939

(22) Filed: Nov. 19, 2024

Related U.S. Application Data
(60) Provisional application No. 63/600,872, filed on Nov. 20, 2023.

(51) Int. Cl. A61B 6/03; A61B 6/00; A61B 6/46

(52) U.S. Cl. CPC A61B 6/03; A61B 6/4441; A61B 6/465

(57) ABSTRACT

Proposed is an X-ray imaging apparatus that, in displaying tomographic images, allows users to quickly and intuitively check the tomographic images they need. The apparatus includes C-arm part including a C-shaped arm, and an X-ray generator and an X-ray detector disposed oppositely at both ends of the C-shaped arm with a subject in between and configured to emit and detect X-rays, a driving part configured to move the X-ray generator separately with respect to an end of the C-shaped arm so that the X-ray generator emits X-rays at different angles toward the subject, and a viewer module configured to reconstruct some tomographic images of the region of interest among a plurality of tomographic planes of the subject using X-ray detection results from different angles detected by the X-ray detector, and display the reconstructed images on a screen.

### CROSS REFERENCE TO RELATED APPLICATION

This application claims the benefit of the filing date of U.S. Patent Provisional Application No. 63/600,872, filed Nov. 20, 2023, as Attorney Docket No.: 810.0159prov, the teaches of which are incorporated herein their entirety by reference.

### BACKGROUND

### Technical Field

The present disclosure relates to an X-ray imaging apparatus in which an X-ray generator and an X-ray detector are respectively disposed at opposite ends of a C-shaped arm.

### Description of the Related Art

An X-ray machine is a device that emits X-rays toward a subject, obtains an X-ray image from the X-rays that travel through the subject to display the internal structure of the subject.

An example of the X-ray machine is an X-ray fluoroscopy apparatus, commonly called a C-arm, which is used in operating rooms, emergency rooms, or during other clinical procedures. As an example, the C-arm X-ray machine consists of a mobile base, a multi-joint arm connected to the mobile base, and a C-shaped arm connected to the multi-joint arm. Additionally, an X-ray generator and an X-ray detector are respectively installed at opposite ends of the C-shaped arm to capture an X-ray fluoroscopy image of an object placed therebetween.

The C-arm X-ray machine is mainly used to acquire two-dimensional X-ray fluoroscopy images of a subject. Meanwhile, in order to check the exact location of a lesion, surgical progress, location of a surgical instrument, and degree of fastening during surgery, tomographic images may be required. However, it is difficult to move a patient to an imaging room equipped with a CT imaging system during surgery to obtain tomographic images. A C-arm X-ray machine with a tomosynthesis function, which allows multiple tomographic images of a subject to be acquired through X-ray imaging over a limited angular range can be an alternative.

Meanwhile, to effectively utilize multiple tomographic images during surgery or similar situations, it is necessary to enable users to quickly and intuitively check the tomographic images they need. However, in a conventional C-arm X-ray machine, a plurality of tomographic images is arranged and displayed on the screen in the form of thumbnails or small previews, and when a user selects one of the images, the selected image is enlarged and displayed, or a random tomographic image is displayed on the screen and the tomographic image above or below it, that is, the position of the tomographic image relative to the direction of X-ray emission, is sequentially displayed according to the user's operation (mouse scrolling, etc.). In the former case, it is difficult to intuitively recognize the relative position of a tomographic image or the differences between adjacent tomographic images, whereas in the latter case, there is the inconvenience of having to keep scrolling until the tomographic image the user wants is displayed. Accordingly, an X-ray imaging device that enables intuitive recognition of the position of a tomographic image and allows users to easily and quickly find the desired tomographic image is required.

### Document of Related Art

(Patent Document 1) Korean Patent Application Publication No. 10-2016-0071938
(Patent Document 2) Korean Patent Application Publication No. 10-2013-0058633
(Patent Document 3) U.S. Patent Application Publication No. 2020-0085390

### SUMMARY

Accordingly, the present disclosure has been made keeping in mind the above problems occurring in the related art, and the present disclosure is intended to provide an X-ray imaging apparatus that is easy to be moved and used in a tight space such as an operating room and is capable of performing tomosynthesis imaging, and displays multiple tomographic images obtained through the tomosynthesis imaging on the screen, allowing users to quickly and intuitively check the tomographic images they need.

In order to achieve the above objective, according to an aspect of the present disclosure, there is provided an X-ray imaging apparatus including: a C-arm part including a C-shaped arm, and an X-ray generator and an X-ray detector disposed oppositely at both ends of the C-shaped arm with a subject in between, and configured to emit and detect X-rays; a driving part configured to move the X-ray generator separately with respect to an end of the C-shaped arm so that the X-ray generator emits X-rays at different angles toward the subject; and a viewer module configured to reconstruct some tomographic images of the region of interest among a plurality of tomographic planes of the subject using X-ray detection results from different angles detected by the X-ray detector, and display the reconstructed images on a screen.

The viewer module may first display a tomographic image with the largest area of a high-frequency region among the tomographic images.

The viewer module may first display a tomographic image in which metal is present among the tomographic images.

The apparatus may further include an input part configured to receive user input for operating the viewer module, wherein the viewer module may sequentially display the tomographic images according to heights of the tomographic planes according to the user input through the input part.

In this case, the viewer module may display a tomographic image display window on the screen and display the tomographic images within the tomographic image display window.

At this time, the viewer module may further display an indicator indicating a position of a tomographic plane of a tomographic image displayed in the tomographic image display window.

In addition, the viewer module may further display an additional image display window on the screen, and display one of the tomographic images or an X-ray image different from the tomographic images for the subject in the additional image display window, wherein the X-ray image different from the tomographic images may be a fluoroscopic image, multiple tomographic images in a weight bearing state, and multiple tomographic images in a non-weight bearing state.

According to the present disclosure, provided is an X-ray imaging apparatus that is easy to be moved and used in a narrow space such as an operating room and is capable of performing tomosynthesis imaging, and displays multiple tomographic images obtained through the tomosynthesis imaging on the screen, allowing users to quickly and intuitively check the tomographic images they need.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 shows an X-ray imaging apparatus according to an embodiment of the present disclosure;

FIGS. 2 and 3 each show a part of an X-ray imaging apparatus according to an embodiment of the present disclosure;

FIG. 4 shows the configuration of a controller according to an embodiment of the present disclosure;

FIG. 5 shows a tomographic image display method according to some embodiments of the present disclosure;

FIG. 6 shows a tomographic image display method according to some other embodiments of the present disclosure;

FIG. 7 shows a tomographic image display window on the screen according to an embodiment of the present disclosure; and

FIG. 8 shows a tomographic image display screen according to an embodiment of the present disclosure.

### DETAILED DESCRIPTION

Hereinafter, embodiments of the present disclosure will be described in detail with reference to the drawings. The technical idea of the present disclosure may be more clearly understood through the embodiments. The present disclosure is not limited to the embodiments described below.

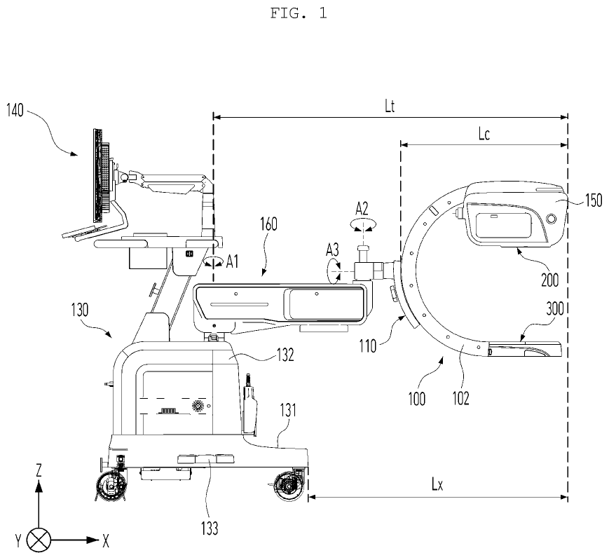

FIG. 1 shows an X-ray imaging apparatus according to an embodiment of the present disclosure.

In FIG. 1 and the following drawings, the x-axis direction represents the front of the X-ray imaging apparatus, the y-axis direction represents a direction perpendicular to the x-axis on the plane where the X-ray imaging apparatus is placed, and the z-axis direction represents a direction perpendicular to the plane.

The X-ray imaging apparatus according to this embodiment of the present disclosure largely include a main body part 130, a C-arm part 100, and an arm part 160 connecting the main body part 130 and the C-arm part 100. A mobile base 131 is provided at the lower part of the main body part 130 to support the entire load of the apparatus and to be movable. Additionally, the main body part 130 may have a built-in controller 140 such as a console PC responsible for apparatus control and image processing. The controller 140 may be disposed above the main body part 130 and may include a display that outputs an X-ray image, etc., an input part where user commands are input, a memory for storing projection data that is the X-ray detection result detected by an X-ray detector described later, and a viewer module that reconstructs projection data stored in the memory into a predetermined X-ray image and displays the image through the display.

The viewer module of the X-ray imaging apparatus according to this embodiment may reconstruct and provide a plurality of tomographic images of a subject using projection data acquired from different angles on the subject obtained through tomosynthesis imaging by means of the C-arm part 100.

The mobile base 131 may include wheels arranged at each of the four corners for stable movement and a frame supporting the wheels.

The rear end of the arm part 160 is connected to the main body part 130 and the front end of the arm part 160 is connected to the C-arm part 100. To be specific, the arm part 160 is connected to the main body part 130 so as to rotate up, down, left, and right within at least a predetermined angle range about a first axis A1 parallel to the z-axis. The front end of the arm part 160 and the C-arm part 100 are connected through a C-arm supporter 110.

The C-arm part 100 includes an X-ray generator 200 (hereinafter referred to as generator), an X-ray detector 300 (hereinafter referred to as detector), and a C-shaped arm 102 with the X-ray generator 200 and the X-ray detector 300 respectively disposed at opposite ends.

The C-arm supporter 110 has one side thereof supporting the C-arm part 100 so as to slide along the C-shaped curved surface of the C-shaped arm 102, and has the other side thereof rotatably connected to the front end of the arm part 160 about a second axis A2. One side of the C-arm supporter 110 that is slidably connected to the C-shaped arm 102 may be rotatably connected to the other side of the C-arm supporter 110 connected to the arm part 160 about a third axis A3.

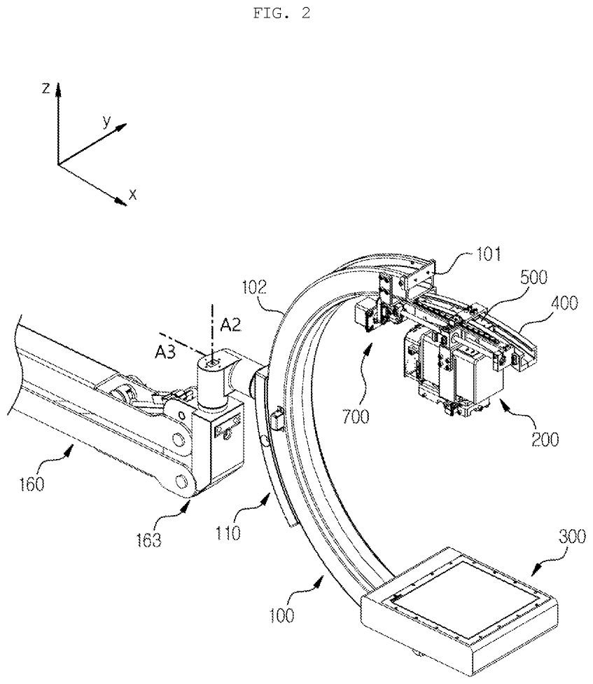

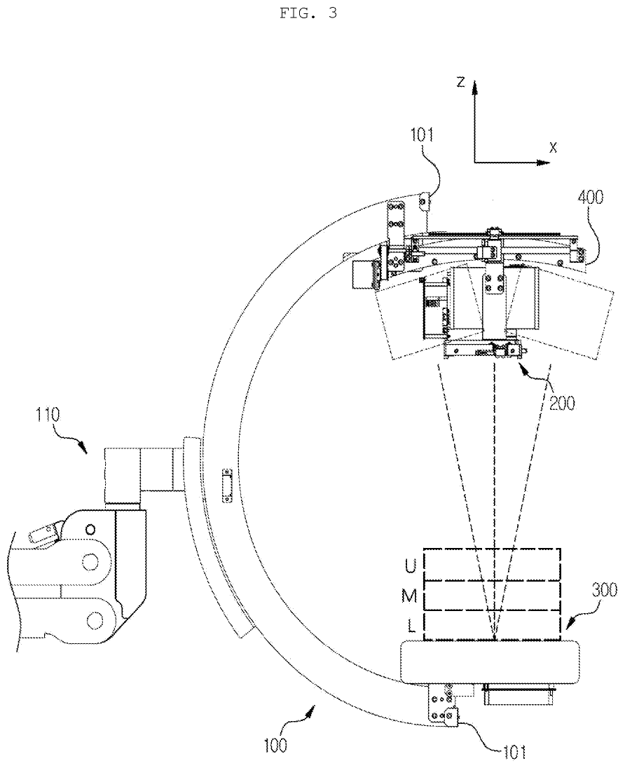

FIGS. 2 and 3 each show a part of an X-ray imaging apparatus according to an embodiment of the present disclosure.

FIGS. 2 and 3 show a generator part casing 150 removed so that the principle of tomosynthesis imaging using the X-ray imaging apparatus according to this embodiment can be understood.

In the C-arm part 100, an arcuate guide rail 400 is disposed at one end of the C-shaped arm 102 in the front-back direction of the extension line, the generator 200 may move in the front-back direction along the arcuate guide rail 400. To this end, the X-ray imaging apparatus according to this embodiment may include a driving part 700 that provides power to move the generator 200.

The driving part 700 may include a motor. In addition, the driving part 700 converts the rotational motion of the motor into linear motion and transmits the linear motion back to a moving block 500 disposed within the arcuate guide rail 400 to convert the linear motion into arc-shaped trajectory motion. The generator 200 is fixed to the moving block 500 and moves along the arcuate guide rail 400. At this time, the generator 200 moves while maintaining an equal distance from the detector 300, thereby emitting X-rays at different angles toward the subject place between the generator 200 and the detector 300. The detector 300 may perform tomosynthesis imaging by detecting X-rays that pass through the subject at different angles. However, the above-described power transmission method is not limited and may be implemented in various ways, such as directly transmitting the power of the drive motor to the motion of an arc-shaped trajectory using a flexible power transmission means such as a chain or timing belt.

In FIG. 3, the outline of the generator 200 indicated by a thin dotted line represents the maximum range over which the generator 200 may move, and the thick dotted line indicates the change in the irradiation angle of an X-ray beam when the generator 200 is located at the front/rear ends and center of the arcuate guide rail 400.

In this way, when the generator 200 moves along the guide rail 400 and emits X-rays at various angles toward the subject, the detector 300 may obtain projection data acquired from various angles for the subject, and the viewer module may provide multiple tomographic images of the subject by reconstructing the projection data acquired from various angles using a predetermined image processing algorithm.

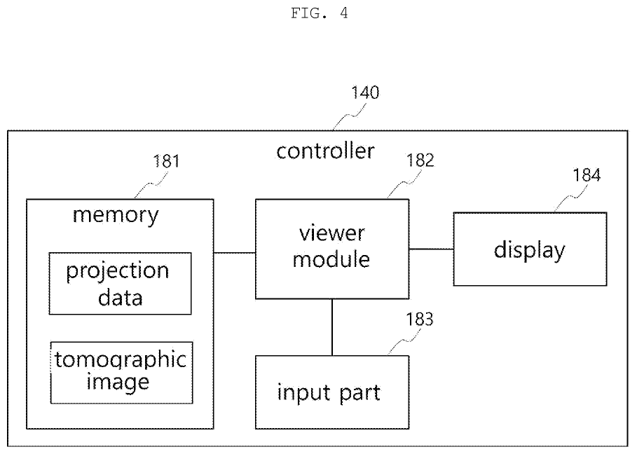

FIG. 4 shows the configuration of a controller according to an embodiment of the present disclosure.

The controller 140 may include: a memory 181 that stores X-ray projection data, which is the detection result detected by the X-ray detector, and an X-ray image reconstructed based on the projection data; a viewer module 182 that reconstructs a plurality of tomographic images or real-time fluoroscopic images using the projection data and displays the images in a predetermined form through the display 184; and an input part 183 that receives user input for controlling the viewer module 182. At this time, the viewer module 182 may reconstruct a plurality of tomographic images and/or fluoroscopic images and display the images on the display 184, since there is no significant difference in the method of displaying fluoroscopic images compared to the conventional method, the explanation will focus on multiple tomographic images.

The viewer module 182 reconstructs tomographic images at different depths facing the direction of X-ray emission using projection data acquired from different angles for the subject. The X-ray imaging apparatus according to this embodiment may, for example, provide a plurality of tomographic images, each with a certain thickness, from above of the light-receiving surface of the detector 300, for example, 250 tomographic images with an interval of 0.7 mm. The viewer module may divide the plurality of tomographic images into several regions, for example, three regions according to the height from the detector 300 for each region of interest of an object to be imaged. The regions of these tomographic images may be divided in relation to the type of main imaging object.

The main imaging object of the X-ray imaging apparatus such as this embodiment include a hand, a foot, a knee, etc. According to an object, the viewer module may group the tomographic images into a lower region L (e.g., height 0-5.8 cm, slices 1-82) mainly corresponding to the focal position of the hand; a middle region M (e.g., height 5.8-11.6 cm, slices 83-166) mainly corresponding to the focal position of the foot; and an upper region U (e.g., height 11.6-17.4 cm, slices 167-250) mainly corresponding to the focal position of the knee.

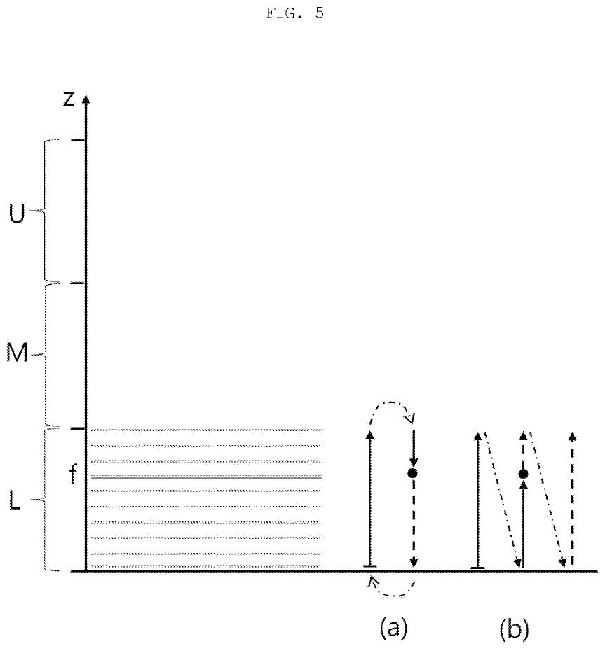

FIG. 5 shows a tomographic image display method according to some embodiments of the present disclosure.

The viewer module of the X-ray imaging apparatus according to an embodiment of the present disclosure may be configured to reconstruct and display at least one group of tomographic images corresponding to the region of interest among the plurality of tomographic image groups described above according to the object. The object, and the tomographic image region to be reconstructed accordingly, that is, the region of interest, may be selected through user input, or may be selected automatically through image recognition or detection results by a separate sensor.

First, when a user selects the hand imaging mode, the X-ray imaging apparatus according to this embodiment reconstructs tomographic images of the lower region L through a tomosynthesis imaging sequence. The left part of FIG. 5 is a simplified representation of multiple reconstructed tomographic images. On the other hand, when the foot imaging mode is selected, tomographic images from the lower region L to the middle region M, preferably the middle region M, are reconstructed. When the knee imaging mode is selected, tomographic images from the lower region L to the upper region U, preferably the upper region U, will be reconstructed.

At this time, in foot imaging mode, the foot that is the object to be imaged is located from the lower region L to the middle region M, and thus tomographic images from the upper region U are unnecessary. Accordingly, the viewer module may reconstruct tomographic images from the lower region L to the middle region M. However, because the clinical region of interest is mainly the middle region M, only tomographic images from the middle region M may be reconstructed. For the same reason, in the knee imaging mode, since the knee that is the object to be imaged is disposed from the lower region L to the upper region U, tomographic images from the lower, middle, and upper regions L, M, and U may be reconstructed. However, because the clinical region of interest is mainly the upper region U, the viewer module may reconstruct only tomographic images from the upper region U.

According to the embodiment (a), the viewer module of the X-ray imaging apparatus according to the present disclosure may sequentially display the tomographic image of the tomographic plane corresponding to the lowest height in the lower region L in the tomographic image display window in order of height. When there is no user input until all of the 1st to 82nd tomographic images are displayed, the tomographic image corresponding to the highest position tomographic plane may be continuously displayed in reverse During this continuous playback, if the user finds a tomographic plane f that he or she wants to check and selects the corresponding tomographic plane (e.g., by clicking the mouse or stepping on the pedal), the viewer module stops continuous playback and displays the selected tomographic image in the display window. When there is no user selection input, the tomographic image in the forward direction, where the height of the tomographic plane increases, and in the reverse direction, where the height of the tomographic plane decreases, may be played repeatedly and continuously. Even if there is a user input, the user may choose to resume continuous playback.

According to the embodiment (b), after the tomographic image of the highest tomographic plane is displayed through continuous playback in the forward direction starting from the lowest tomographic plane, continuous playback from the lowest tomographic plane in the forward direction may be repeated. In this case as well, when there is a user's selection input for the specific tomographic plane f, continuous playback stopped, and continuous playback may also be resumed according to the user's input.

In this case, although in the embodiments (a) and (b), continuous playback is performed in an order starting from the tomographic plane with the lowest height and increasing in height, conversely, continuous playback may be started in the order starting from the tomographic plane with the highest height and gradually decreasing in height.

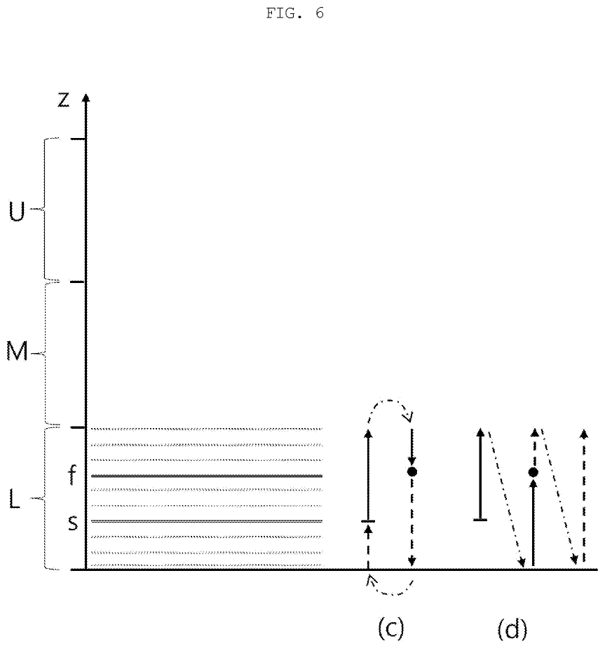

FIG. 6 shows a tomographic image display method according to some other embodiments of the present disclosure.

According to the embodiment (c), continuous playback may be started in the forward direction from a starting tomographic plane s determined according to a preset standard among the reconstructed tomographic images of the lower region L, and when the highest tomographic plane is reached, continuous playback is performed in reverse order, and continuous playback may be stopped if there is a user input for the specific tomographic plane f.

According to the embodiment (d), continuous playback may be started in the forward direction from the starting tomographic plane s, and when the highest tomographic plane is reached, continuous playback in the forward direction may be repeated again from the lowest tomographic plane. In this case as well, if the user selects the specific fault plane f, continuous playback is stopped.

The starting tomographic plane s may be, for example, a tomographic image determined to be the clearest among the tomographic images of the group reconstructed according to the user's selection of imaging mode. As an example, the X-ray imaging apparatus may be configured to select the tomographic plane with the largest area of the high-frequency region as the starting tomographic plane s by using an algorithm for calculating the area of the high-frequency region where the spatial frequency is higher than a reference value (high image sharpness). Such configuration allows users to more quickly find a tomographic image that is well-focused on the area they want to check.

The starting tomographic plane s may be tomographic image in which metal is determined to be present among the group of tomographic images reconstructed according to the user's selection of the imaging mode. As an example, tomosynthesis imaging by the X-ray imaging apparatus according to this embodiment may be used when inserting metal pins, etc. for fracture treatment. Thus, the X-ray imaging apparatus may be configured to select one of the tomographic images in which metal is detected based on projection data as the starting tomographic plane s, for example, to select the tomographic image in which the metal is first or last detected, the area of the metal is largest, or the metal is in focus as the starting tomographic plane s. Such configuration allows users to more quickly find the tomographic image they want to check.

In this case, although in the embodiments (c) and (d), continuous playback is started in the order in which the height of the tomographic plane gradually increases, conversely, continuous playback may be started in the order in which the height of the tomographic plane gradually decreases.

Meanwhile, although the embodiments (a), (b), (c) and (d) are about hand imaging mode and the lower region L is used as an example, in the case of foot imaging mode, the above display method may be applied from the lower region L to the middle region M, preferably to the middle region M, and in the case of knee imaging mode, the above display method may be applied from the lower region L to the upper region U, preferably to the upper region U.

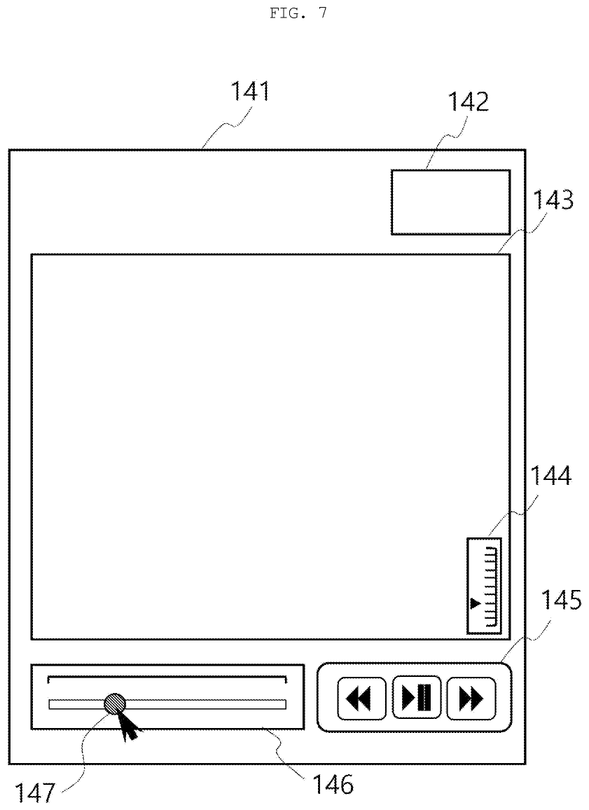

FIG. 7 shows a tomographic image display window on the screen according to an embodiment of the present disclosure.

According to this embodiment, a tomographic image display window 141 displayed on the screen by means of the viewer module of the X-ray imaging apparatus includes an imaging information display part 142 and a tomographic image display part 143. The tomographic image display part 143 may include an indicator part 144 for indicating the position of the tomographic plane currently being displayed. The indicator part 144 may be placed within the tomographic image display part 143, and in this case, may be displayed translucently.

A button-type control part 145 may be provided within the tomographic image display window 141 so that the user can stop, resume, fast-forward, rewind, etc. the continuous playback of multiple tomographic planes corresponding to the tomographic images. In this case, the fast-forward or rewind button may speed up continuous playback by reducing the display time for each tomographic plane in the forward or reverse direction, or may be used to skip several to dozens of tomographic planes for displaying.

In addition, the tomographic image display window 141 may include a bar-shaped control part 146 that displays a position indicator 417 of the tomographic plane being displayed on the tomographic image display part 143 on a bar, and that allows the user to immediately change the position of the displayed tomographic plane even during continuous playback by moving the position indicator 417 using an input method such as a mouse drag.

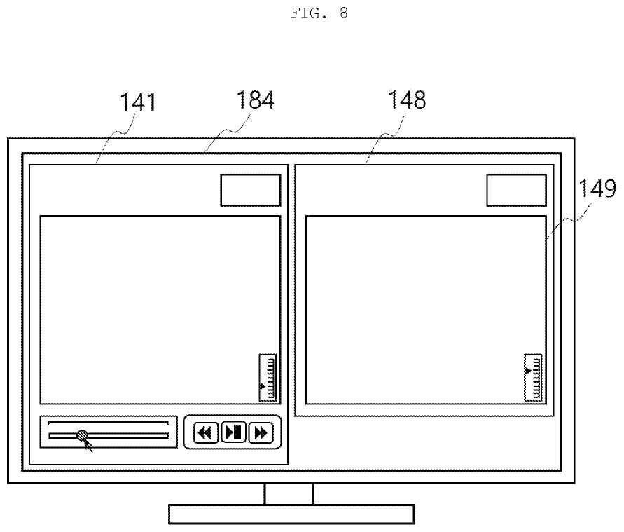

FIG. 8 shows a tomographic image display screen according to an embodiment of the present disclosure.

The display 184 screen of the X-ray imaging apparatus according to an embodiment of the present disclosure may further include an additional image display window 148 together with the tomographic image display window 141. As an example, the additional image display window 148 may include a selected image display part 149 that displays the tomographic plane corresponding to the tomographic image selected by the user using various control parts in the tomographic image display window 141 or a separate input means such as a pedal. The above-described indicator part may also be included in the selected image display part 149. Meanwhile, in the additional image display window 148, in addition to the tomographic plane corresponding to the tomographic image selected by the user, X-ray images produced by other imaging modalities, including real-time fluoroscopic images, may be displayed together. Particularly, one of the tomographic image display window 141 additional image display window 148 may display and the multiple tomographic images in the weight bearing state, and the other may display multiple tomographic images in the non-weight bearing state.

What is claimed is:

1. An X-ray imaging apparatus comprising:
a C-arm part configured to include a C-shaped arm, and an X-ray generator and an X-ray detector disposed oppositely at both ends of the C-shaped arm with a subject in between;
a driving part configured to move the X-ray generator from an end of the C-shaped arm in a predetermined range in the forward and backward direction so that the X-ray generator emits X-rays at different angles toward the subject;
a viewer module configured to reconstruct a plurality of tomographic images of the subject using X-ray detection results from different angles detected by the X-ray detector, and display the reconstructed images on a screen; and
an input part configured to receive information about the type of the subject,
Wherein the viewer module reconstructs tomographic images of a region of interest predetermined for the subject according to information about the type of the subject received through the input unit and displays them on the screen.

2. The apparatus of claim 1, wherein the viewer module first displays a tomographic image with a largest area of a high-frequency region among the tomographic images.

3. The apparatus of claim 1, wherein the viewer module first displays a tomographic image in which metal is present among the tomographic images.

4. The apparatus of claim 1, wherein the input part further receives user input and sequentially displays the tomographic images according to heights of the tomographic planes according to the user input through the input part.

5. The apparatus of claim 4, wherein the viewer module displays a tomographic image display window on the screen and displays the tomographic images within the tomographic image display window.

6. The apparatus of claim 5, wherein the viewer module further displays an indicator indicating a position of a tomographic plane of a tomographic image displayed in the tomographic image display window.

7. The apparatus of claim 5, wherein the viewer module further displays an additional image display window on the screen, and displays one of the tomographic images or an X-ray image different from the tomographic images for the subject in the additional image display window,
wherein the X-ray image different from the tomographic images is a fluoroscopic image, multiple tomographic images in a weight bearing state, and multiple tomographic images in a non-weight bearing state.
